(19) United States
(12) Patent Application Publication
(10) Pub. No.: US 2004/0126384 A1
(43) Pub. Date: Jul. 1, 2004

(54) Combination of IgY against dental caries

(76) Inventors: Rong Jian Yang, Wan Chai (HK); Shing Paau, Causeway Bay (HK)

Correspondence Address: Raymond Y. Chan, Suite 128, 108 N. Ynez Avenue, Monterey Park, CA, 91754, US

(21) Appl. No.: 10/737,332

(22) Filed: Dec. 15, 2003

Related U.S. Application Data
(62) Division of application No. 09/684,794, filed on Oct. 10, 2000, now abandoned.

(51) Int. Cl. A61K 39/00; A61K 39/38

(52) U.S. Cl. 424/184.1

(57) ABSTRACT

For the purpose of preparing the immunoglobulin of yolk (IgY) against dental caries bacteria and the combination, Streptococcus mutans type c and type d are used as antigen bacteria, the antibody is purified by water dilution, DEAE-Sephadex A50 and Sephadex G200 chromatography. It is low cost of production, high titer, strong resistance to osmotic pressure, and high immune activity and wide range of cross-reaction to streptococcus mutans. Present invention provides the combination preventing dental caries wherein the effective components are IgY and antiseptic features in safe use and effective prevention; The combination is effectively preventing dental caries.

### CROSS REFERENCE OF RELATED APPLICATION

This is a divisional application of a non-provisional application, application Ser. No. 09/684,794, filed Oct. 10, 2000.

### BACKGROUND OF THE PRESENT INVENTION

1. Field of Invention

The present invention relates to preparation of immunoglobulin from hen yolk, and more particularly to IgY against dental caries bacteria and the combination preventing dental caries wherein the IgY and antiseptic are effective components.

2. Description of Related Arts

It is well known that streptococcus mutans are major dental caries bacteria. There are two measures of passive immunization to streptococcus mutans at this moment.

(1) Cows or hens are immunized with single or mixed streptococcus mutans as antigen. The antibodies are extracted from milk or yolk. Then, passive immunization is taken place.

(2) Cows or hens are immunized with glucosyl transferase of streptococcus mutans as antigen. The following steps are as same as those of the first measure.

There are, however, problems for available preparative technique of antibody:

(1) high cost of antibody preparation, especially, difficulty of antigen extract while glucosyl transferasse is used as antigen.

(2) low titer of antibody while the highest titer is only 1:320 by existing preparative technique.

(3) The yolk could not be comprehensively used.

### SUMMARY OF THE PRESENT INVENTION

The main objective of present invention is to provide a preparative method of IgY against dental caries, which reduces cost of production, elevates titer of antibody, enables yolk to be utilized in multi-propose, simultaneously, provides dental caries preventing combinations where IgY of the present invention and antiseptic are effective components.

The preparative method of IgY against dental caries of the invention includes the following steps:

Streptococcus mutans type c and type d are separately grown in BHI or TTY culture medium for 2-3 days and centrifuged to collect the bacteria. The bacteria are washed 4-6 times with 0.05-0.2 M of phosphate buffered saline, pH 6-7, heated at 50-60° C. for 25-35 minutes. To prepare antigens, mix streptococcus mutans type c and type d by ratio 1:1-2:1, add Freund's adjuvant equal to total volume of both bacteria, and then treat with high speed homogenize machine.

The best ratio of type c and type d mixture is 2:1.

The hens are immunized by three hypodermic or wing vein injections, 1.0 ml (1×10⁹/ml) of streptococcus mutans each time, at 2 weeks intervals. Yolks are taken out by sieve, stirred even, and diluted by adding 4-6 fold of distilled water. Adjust pH to 4.5-6.5, stand at 3-5° C. for 20-30 hours, and centrifuge at high speed for 20-30 minutes. The supernatant is ultrafiltrated, followed by 0.22 μm membrane filtration to eliminate bacteria and lyophilization. This is crude IgY extract against dental caries.

The crude extract is applied on DEAE-Sephadex A50 column and eluted with phosphate buffer containing 0.03-0.1M of NaCl by gradient elution followed by pouring protein peaks, estimating antibody activity with “ELISA”, and adjusting active eluates to 20 mg protein/ml.

Obtained eluates are applied on Sephadex G200 column, and eluted with phosphate buffer containing 0.05-0.2M of NaCl by gradient elution followed by pouring protein peaks, estimating antibody activity with “ELISA”, eliminating bacteria by 0.22 μm membrane filtration, and lyophilizing. This is purified IgY against dental caries bacteria.

The best concentrations of NaCl in phosphate buffer eluants for “DEAE-Saphadex A50” and “Sephadex G200” are 0.07M and 0.1M respectively.

Based on the following results, the present invention chooses streptococcus mutans type c and type d as the antigen bacteria.

Streptococcus mutans have serotype a, b, c, d, e, f, g, and h, etc. in accordance with serum typing. In oral cavity, however, type c and type d account for about 60-90% and 10% respectively. The others are very low, therefore, type c and type d are major serotype bacteria causing dental caries. Choosing type c and type d as antigen bacteria advantages in either avoiding reducing efficiency of major serotype bacteria when mixture of multi-serotype bacteria is used as antigens, or avoiding narrow range of immunological cross reaction when single serotype bacteria is used as antigens. Moreover, the investigations indicate that protein antigen A and B can be extracted from cell wall of streptococcus mutans of oral cavity, type c has both protein A and B, type b has only protein A, and type a, d, e, and g have only protein B. Thus, type c and d as antigens insure that the antibodies have wide range of cross reaction.

The present invention adopts water dilution to extract IgY, which comprehensively utilizes the yolks with low cost, simple technology and no environmental pollution.

IgY of the present invention has reached PAGE purity with 180 kD of molecular weight by SDS-PAGE.

The experiments showed that the IgY keeps its activity during 90 minutes at 65° C. Its activity has no significant change after 8 hours at 37° C. but rapidly decreased and inactivated at pH 2.0 or pH 12.0. The IgY is resistant to osmotic pressure, for instance, tolerant to 40% sucrose.

The IgY of the present invention can effectively inhibit agglutination of streptococcus mutans by indirect hemagglutination test with the titer 1:512 and obviously inhibit adhesion of the bacteria until it is diluted to 1:8. Animal experiments take that IgY can effectively prevent occurrence of dental caries by feeding IgY rats infected with streptococcus mutans. The bacteria number in bacteria plaques reduces 70-80%. The results of contrast experiment of dental caries formation in rats is expressed in the following table:

| | Control | Experiment | p Value |
|---|---|---|---|
| I | 47 | 19 | <0.01 |
| II | 25 | 0.57 | <0.01 |
| III | 8 | 0 | <0.01 |

I: only damage of enamel;
II: damaged ¼ of denting;
III: damaged through dentine. Dental caries score by Keyes method.

The difference between two groups is very significant (p<0.01).

The dental caries preventing combination means that the combination's effective components are IgY of the present invention and antiseptic, the later is, at least, one of both potassium sorbate and sodium benzoate. The combination may be either a product for oral cavity use, for example, toothpaste, buccal liquid, mouthwash, or food, for instance, chewing gum, chocolate, ice cream, milk (powder), bean milk (powder), etc. Additive amount of the IgY usually is 0.05-0.2%. Potassium sorbate or sodium benzoate is 0.005-0.02%.

The combination can be packaged in pocket atomizer as liquid product is used in oral cavity, and in sucking bottle as food for serving.

Coordinated with decontaminaters and ozostomia preventer, diverse products can be manufactured of the IgY for prevention and treatment. Also, it can be added in refreshing agent of oral cavity to enhance their function preventing dental caries.

The IgY of the invention is low cost of preparation and production, high titer of antibody, resistant to osmotic pressure, strong immunological activity and wide range of cross-reaction to streptococcus mutans. The combination of the present invention features in small amount of IgY, safe use, effective prevention, and treatment, etc. It can effectively prevent occurrence of dental caries.

### DETAILED DESCRIPTION OF THE PREFERRED EMBODIMENT

The invention will be described further by the following samples:

Sample 1 Streptococcus mutans type c and type d are separately cultivated in BHI medium at 37° C. for 48 hours, followed by collecting bacteria with centrifugation, at 4000 rpm for 10 minutes, washing 5 times with 0.2M of phosphate buffered saline, pH 6.0, and heating at 50° C. for 25 minutes. Each of type c and type d is adjusted to 2×10⁹/ml of the suspensions. Mix equal volume of type c and type d. Then, Freund's adjuvant equal to total volume of type c and type d is added in it, and then homogenized at high speed. This is a streptococcus mutans antigen.

The hens are immunized by three hypodermic injuctions, 1.0 ml (1×10⁹/ml) of bacteria antigens each time at 2 weeks intervals. Eggs are collected from 20^{th} day after first immunization and sterilized with 75% alcohol. The yolks are taken out with sieve and stired to even, diluted with 5 fold distilled water, adjusted pH to 6.0, standed at 3° C. for 24 hours, and then centrifuged at 8000 rpm for 25 minutes. The supernatant is concentrated by ultrafiltration, eliminating bacteria and lyophilization. This is crude IgY extract against dental caries bacteria.

Three milliliter (10 mg/ml) of crude IgY extract are applied on “DEAE-Sephadex A50” column (2.5×35 cm), eluted with pH 7.0, 0.01 M of phosphate buffer containing 0.07M of NaCl, 20 ml/h. 5.0 ml each fraction. The protein peaks are poured. Antibody activity are estimated with “ELISA”. Active eluates are poured. Adjusted to 20 mg protein/ml. Then, 1.5 ml of it is applied on “Sephadex G200” column (2.0×65 cm) and eluted with pH 7.0, 0.01 M of phosphate buffer containing 0.1M of NaCl, 8.0 ml/h. 5.0 ml each fraction. The protein peaks are poured and estimated for antibody activity with “ELISA”. Active eluates are poured, bacteria-eliminated with 0.22 μm membrane filtration, and then lyophilized. This is purified IgY against dental caries bacteria.

Sample 2 Streptococcus mutans type c and type d are separately cultivated in TTY medium at 37° C. for 48 hours, collected by centrifugation at 4000 rpm for 10 minutes, washed with pH 6.5, 0.15M of phosphate buffered saline 5 times, and heated at 65° C. for 25 minutes. Then, make type c and type d suspensions, 2×10⁹/ml each. Mix 2:1 volumes of type c and type d suspensions to get mixture (2×10⁹/ml) of them. Add Freund's adjuvant equal to the volume of the mixture. Treat it with high speed homogenize machine to get streptococcus mutans antigens.

To get crude IgY extract against dental caries bacteria, immunize hens by three injections in wing vein, 1.0 ml (1×10⁹/ml) of antigens each time, at 2 weeks intervals. Collect eggs from 20^{th} day after first injection. Sterilize the eggs by 75% alcohol. Take yolks out by sieve. Stir them even. Dilute with 6 fold volume of distilled water. Adjust pH to 5.5. Stand at 4° C. for 24 hours. Centrifuge at 8000 rpm for 25 minutes. Concentrate the supernatant by ultrafiltration, eliminating bacteria and lyophilization.

To get purified IgY against dental caries bacteria, apply 4.0 ml (10 mg/ml) of crude IgY on “DEAE-Sephadex A50” column (2.5×35 cm), elute with pH 7.0, 0.01 M of phosphate buffer containing 0.06M of NaCl, 20 ml/h, 5.0 ml each fraction, pour each peask, estimate antibody activity with “ELISA”. Keep the active eluates, eliminate bacteria by 0.22 μm membrane filtration and lyophilize. Preparation of product of the combination preventing dental caries:

Sample 3 Preparation of IgY Buccal Liquid

Take 2.0 g of the IgY, 0.15 g of potassium sorbate, 0.8 g of aspartame, and 0.15 g of menthol, and 0.4 ml of apple essence. Add menthol into 100 ml of distilled water and dissolved at 60° C. Other solid components are dissolved in 450 ml of distilled water, combine both solutions, and then add distilled water to 1 000 ml.

Sample 4 Preparation of Chewing Gum

Take 2.0 g of the IgY, 0.15 g of potassium sorbate, 0.8 g of aspartame, 0.15 g of menthol, 0.4 ml of apple essence, 10.08 g gum base and 5.08 g of CM-cellulose, and then add substrate material to 1 000 g.

Sample 5 Preparation of IgY Toothpaste

Take 0.1 g of the IgY, 0.015 g of potassium sorbate, 0.015 g of sodium benzoate, 10.0 g of glycerol, 8.0 g of sorbitol, 2.0 g of CM-cellulose, 1.3 g of sodium trehalate, 1.8 g of sodium lauryl sulfate, 0.015 g of menthol, 0.015 g of aspartame, 0.05 ml of strawberry essence, 47.8 g of calcium phosphate 2H₂O. Swell CM-cellulose to dissolve followed by orderly adding other components. Stir thoroughly. Add distilled water to 1 000 ml. Then, stir until it becomes paste.

Sample 6 IgY Tooth-protecting Paste

Take 0.1 g of the IgY, 0.01 g of sodium benzoate, 8.0 g of beeswax, 10.0 g of stearic acid, 2.0 g of monostearyl glyceride, 10.0 g of glycerol, 1.0 g of CM-cellulose, 0.01 g of menthol, 0.05 g of aspartame, 68.80 ml of distilled water and 0.02 g of strawberry essence. Mix beeswax, stearic acid, monotearyl glyceride and glycerol, and heat to 70° C., named solution A. Swell CM-cellulose in 50 ml distilled water to dissolve, orderly and IgY, menthol, aspartame, potassium sorbate, and streaberry essence. Stir thoroughly. Add cooled solution A. Add distilled water to 100 ml and stir until it becomes paste.

Sample 7 Preparation of IgY Nutrient Milk

Add IgY of the present invention and potassium sorbate, which final concentrations are 0.1% and 0.015% respectively, into pasteurized fresh milk, homogenize with sterile homogenizer. Pour into sterile sucking bottles and store at 4° C.

Sample 8 Preparation of IgY Nutrient Milk Powder

Take IgY of the present invention and potassium sorbate, which final concentrations are 0.1% and 0.005% respectively, in pasteurized fresh milk powder. Mix with sterile mixer and package sterily in bags.

Sample 9 Preparation of IgY Nutrient Bean Milk

Add IgY of the present invention and sodium benzoate, which final concentrations are 0.1% and 0.05% respectively, in pasteurized compounded bean milk. Homogenize with sterile homogenier. Pour into sterile sucking bottles and store at 4° C.

Sample 10 Preparation of IgY Nutrient Bean Milk Powder Add IgY of the present invention and sodium benzoate, which final concentrations are 0.1% and 0.005% respectively, in pastuerized bean milk powder. Mix with sterile mixer and package sterily in bags.

What is claimed is:

1. A combination against dental caries bacteria, comprising of effective components composed with IgY to dental caries bacteria and, at least, one of both potassium sorbate and sodium benzoate.

2. The combination, as recited in claim 1, wherein over 0.05% of additive IgY amount, additive amount potassium sorbate and sodium benzoate is 0.005-0.02% respectively.

3. The combination, as recited in claim 2, wherein the additive IgY amount is 0.05-0.2%.

4. The combination, as recited in claim 3, wherein as liquid product used for oral cavity is packaged in pocket atomizer for spraying usage.

5. The combination, as recited in claim 3, wherein as liquid food the combination is packaged in sucking bottle.
